(19) United States
(12) Patent Application Publication
(10) Pub. No.: US 2016/0088715 A1
(43) Pub. Date: Mar. 24, 2016

(54) LIGHTING SYSTEM

(71) Applicant: KONINKLIJKE PHILIPS N.V., Eindhoven (NL)

(72) Inventors: JULIAN CHARLES NOLAN, PULLY (CH); ALEXANDER HENRICUS WALTHERUS VAN EEUWIJK, EINDHOVEN (NL); HILBRAND VANDEN WYNGAERT, GROBBENDONK (BE)

(21) Appl. No.: 14/890,978

(22) PCT Filed: Apr. 30, 2014
(86) PCT No.: PCT/EP2014/058867

§ 371 Date: Nov. 13, 2015

(30) Foreign Application Priority Data
May 14, 2013 (EP) 13167572.0

(51) Int. Cl. H05B 37/02; A61N 5/06

(52) U.S. Cl. CPC H05B 37/0227; H05B 37/0218; A61N 5/0618; A61N 2005/0626

(57) ABSTRACT

A system for influencing a being's perceived taste. The system comprises one or more input devices arranged to receive an input, the input corresponding to a desired of affected change in taste. For example the input may comprise a measure of one or more environmental factors taken using one or more sensors in a target environment, where the one or more environmental factors produce a perceived change in the being's taste. Alternatively the input may be indicative of a change specified for a certain product, user or place. The system further comprises one or more lighting devices arranged to output light into the target environment; and a controller configured to control the light output based on the input, in order to produce an influence on the being's perceived taste which at least partially compensates for the perceived change or goes towards producing the specified change.

### TECHNICAL FIELD

The present disclosure relates to the effect of light on perception of taste.

### BACKGROUND

It has been reported that appetite and digestion can be influenced by the colour of ambient light. Further, recent scientific publications indicate that ambient light can influence the perceived taste of food, e.g. sweetness, saltiness or bitterness. Different colour light can influence different perceived taste attributes, with perceived sweetness and fruitiness for example being illustrated by the study cited below. Indeed it is estimated that for wine, red light can increase sweetness levels by 60%.

[1] “Ambient Lighting Modifies the Flavour of Wine”, Daniel Oberfeld, Heiko Hecht, Ulrich Allendorf and Florian Wickelmaier, Department of Psychology, Johannes Gutenberg Universität, 2009, Journal of Sensory Studies, 24(6), 797-832 (http://homepages.uni-tuebingen.de/florian.wickelmaier/pubs/OberfeldHecht2009JSensS.pdf, DOI 10.1111/j.1745-459X.2009.00239.x)

[2] “Wine and Color: Effects of Ambient Light on Taste and Aroma”, Dr Daniel Oberfeld-Twistel(http://www.staffuni-mainz.de/oberfeld/wine2.html)

In addition, it is known that the colour of light can affect the attractiveness of food in retail counters or on a dish, and the colour of the food or beverage being consumed itself also has an effect on taste. For example reference is made to the following.

[3] “Does Food Color Influence Taste and Flavor Perception in Humans?”, Charles Spence, Carmel A Levitan, Maya U Shankar and Massimiliano Zampini, 9 Mar. 2010, Springer Science & Business Media LLC, Chem. Percept. (2010) 3:68-84 (http://psy.fgu.edu.tw/web/wlchou/general_psychology/class_pdf/Advanced%20Perceptual/2011/2011week4_ChengChung_paper.pdf, DOI 10.1007/s12078-010-9067-z)

[4] “The Multisensory Perception of Flavour”, Charles Spence, The Psychologist, vol. 23, no. 9, September 2010, pages 720-723 (http://www.thepsychologist.org.uk/archive/archive_home.cfm/volumeID_{—}23-editionID_{—}192-ArticleID_{—}1720-getfile_getPDF/thepsychologist/0910spen.pdf)

[5] “Hue and Taste Perception”, Daisy E Del Castillo, Sara Rabiee, Joanna Guerrer and Barbara A Drescher, Department of Psychology, California State University (http://www.icbseverywhere.com/Files/TasteOfColor.pdf)

### SUMMARY

Taste is one of the five traditional senses. Taste is the sensation produced when a substance in the mouth reacts chemically with receptors of taste buds. Taste, along with smell (olfaction) and trigeminal nerve stimulation (which also handles touch for texture, also pain, and temperature), determines flavors, the sensory impressions of food or other substances.

Humans perceive taste through sensory organs called taste buds, or gustatory calyculi, concentrated on the top of the tongue. The tongue is covered with thousands of small bumps called papillae, which are easily visible to the naked eye. Within each papilla are hundreds of taste buds, the organ of taste transduction. There are between 2000 and 5000 taste buds that are located on the back and front of the tongue. Others are located on the roof, sides and back of the mouth, and in the throat. Each taste bud contains 50 to 100 taste receptor cells. Taste perception fades with age: On average, people lose half their taste receptors by time they turn 20.

The sensation of taste can be categorized into five basic tastes: sweetness, sourness, saltiness, bitterness, and umami. Taste buds are able to differentiate between different tastes through detecting interaction with different molecules or ions. Sweet, umami, and bitter tastes are triggered by the binding of molecules to G protein-coupled receptors on the cell membranes of taste buds. Saltiness and sourness are perceived when alkali metal or hydrogen ions enter taste buds, respectively.

The basic tastes contribute only partially to the sensation and flavor of food in the mouth—other factors include smell, detected by the olfactory epithelium of the nose; texture, detected through a variety of mechanoreceptors, muscle nerves, etc.; temperature, detected by thermoreceptors; and “coolness” (such as of menthol) and “hotness” (pungency), through chemesthesis.

Other factors can also be relevant to perception of taste, e.g. altitude, air pressure or humidity, users' personal sensitivity to certain tastes, regional differences in perceived taste, or the nature of the food or drink product in question (e.g. a low-salt version of a meal). It may be desirable to influence perceived changes or differences in taste, or otherwise adjust taste to the requirements of a particular scenario.

Accordingly, in one aspect of the present disclosure, there is provided a system for influencing a being's perceived taste. The system comprises one or more input devices arranged to receive an input, the input corresponding to a desired or affected change in taste. For example the input may comprise a measure of one or more environmental factors taken using one or more sensors in a target environment, where the one or more environmental factors affect a change in the being's perception of taste. Alternatively the input may be indicative of a change specified for a certain product, user or place, such as to tune to a particular user's sensitivity to certain tastes, or to tune to the tastes prevalent in a particular geographic region. The system further comprises one or more lighting devices arranged to provide a light output in the target environment, and a controller configured to control the light output. The controller controls the light output in dependence on the input, in order to produce an influence on the being's perceived taste which at least partially compensates for the perceived change or goes towards producing the specified change.

According to further aspects of the disclosure, there is provided a corresponding method of controlling light to influence a being's perceived taste, and a corresponding computer program product configured to perform operations in accordance with any of the system features disclosed herein.

### BRIEF DESCRIPTION OF THE DRAWINGS

For a better understanding of the disclosure herein and to show how embodiments may be put into effect, reference is made to the accompanying drawings in which:

FIG. 1 schematically illustrates a lighting system installed in a target environment, and

FIG. 2 is a schematic block diagram showing components of the lighting system.

### DETAILED DESCRIPTION OF EMBODIMENTS

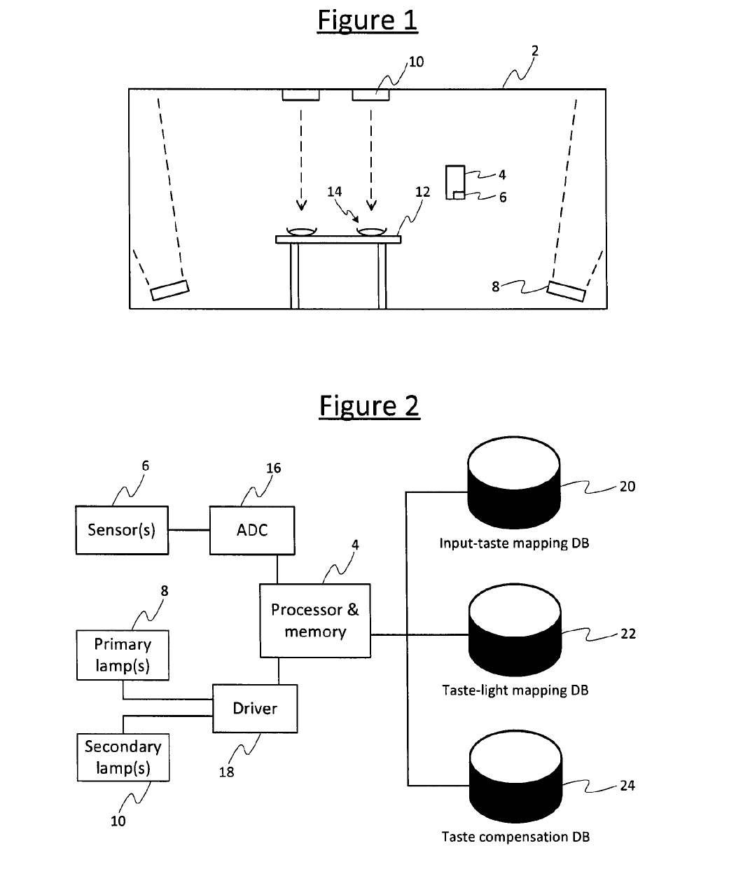

FIG. 1 shows an example of a target environment 2, i.e. a subject environment in which taste is to be influenced in dependence on an input received. The environment may comprise an indoor or outdoor space, e.g. a dining room, a room of a restaurant, or the interior of an aeroplane or other vehicle. The environment 2 may comprise a surface 12 such as a table, providing a location from which an oral product 14 is to be consumed. The product 14 may be food, drink, or some other product to be taken into the mouth such as chewing gum or toothpaste. Note also therefore that consumption is not limited to eating or drinking but may more generally mean to use up orally. The being consuming the product 14 may be a human or other being having the faculty of taste.

The environment 2 is installed with a lighting system comprising one or more lighting devices 8, 10, a controller 4, and one or more input devices 6. The lighting devices 8, 10 are arranged to emit light into the target environment 2. The lighting devices 8, 10 take the form of one or more light sources such as filament bulbs, LED based luminaires or fluorescent tubes. In embodiments, the lighting devices comprise one or more “primary” lighting devices 8 arranged to provide ambient lighting generally within the environment, and one or more “secondary” lighting devices 10 which are discriminately targeted on a location from which an oral product 14 such as food or drink is to be consumed, e.g. a spot on a table. For example the primary, ambient lighting devices 8 may take the form of one or more wall washers or uplighters; and the secondary, target lighting may take the form of one or more spotlights selectively directed onto one or more spots on the table 12 to illuminate one or more dining positions or positions of consumption (and therefore being arranged to illuminate one or more dishes or other products 14).

The controller 4 is configured to control the light output of the one or more lighting devices 8, 10. This may comprise controlling the intensity and/or spectrum (colour) of the emitted light. The controller may be arranged to control the light output of the primary, ambient lighting device(s) 8; and/or the secondary, target lighting device(s) 10. In embodiments it is arranged to control one or both types of lighting 8, 10 independently of the other.

The controller 4 is also arranged to receive an input from one or more input devices 6, where the input has relevance to perceived taste. In embodiments the one or more input devices 6 comprise one or more sensors arranged to dynamically sense the current value of one or more environmental variables in the target environment, and the input comprises a reading of the current value or values, i.e. a measurement of the one or more environmental variables. For example the one or more sensors may comprise an atmospheric pressure sensor and the input may comprise a measurement of a current altitude or air pressure of the target environment 2 taken using the pressure sensor. Alternatively or additionally, the one or more sensors may comprise a humidity sensor and the input may comprise a measurement of a current humidity in the target environment 2 taken using the humidity sensor. In another alternative example, the one or more sensors may comprise a temperature sensor and the input may comprise a measurement of the temperature in the target environment 2 taken using the temperature sensor. In yet another example the one or more sensors may comprise a light sensor arranged to sense natural light such as daylight in the target environment, and the input may comprise a measure of the intensity and/or spectrum of the natural light. Generally any of these or a combination of these sensors may be used, and/or other types of sensor.

Alternatively or additionally the one or more input devices 6 may comprise a detector for detecting the identity of a user, a place, or a product 14. For example, the detector may comprise a camera with facial recognition for recognising the identity of a user, or a detector for detecting an electronic identifier of the user such as an RF tag, or an address or ID of a user terminal of the user, or an ID of an account of the user. As another example the detector may comprise a geographic location technology such as GPS for detecting the location of the target environment 2. In another example, it may comprise a detector for detecting an identifier of the product 14 the taste of which is to be influenced, e.g. a barcode reader for reading a barcode of the product, or a detector for reading an electronic identifier of the product 14 such as an RF tag.

In yet another alternative or additional embodiment, the one or more input devices 6 may comprise a manual user interface such as a touch screen, mouse, touch pad, tracker ball, keyboard or keypad. This may be arranged to allow a user to explicitly enter an identifier of a user, place or product 14, or to explicitly specify a taste requirement such as a desired increase or decrease in sweetness, saltiness, bitterness or sourness.

FIG. 2 gives a block diagram showing various components of the lighting system. The controller 4 may be implemented in code (software) stored on a memory comprising one or more storage media such as electronic or magnetic storage media, the code being arranged for execution on processing apparatus comprising one or more processing units. Alternatively it is not excluded that some or all of the controller 4 is implemented in dedicated hardware circuitry, or reconfigurable hardware circuitry such as an FPGA. In the case where the input device(s) 6 comprises one or more sensors, the controller 4 is coupled to the sensor(s) 6 via an analogue to digital converter (ADC) 16 so as to receive the input measurement in digital form. Further, the controller 4 is coupled to the one or more output devices 8, 10 via a driver 18 comprising one or more driver units for driving the respective lighting devices to emit light with the required output properties.

In embodiments the controller 4 is integrated into a unit installed in the target environment 2, e.g. in a central controller installed at suitable location such as on a wall or in the ceiling, or a controller integrated into one or more of the luminaires 8, 10. In other embodiments, the controller 4 may be implemented at least partially in a mobile device or other user terminal, e.g. as an application running on a smart phone or tablet. In this case the functionality of the controller 4 may be split between the user terminal and a unit installed in the target environment. The unit may be a lighting control unit. For example the mobile terminal may be configured to look up the desired light settings and communicate these to the lighting control unit via a suitable interface such as a wireless interface, e.g. Bluetooth or Wi-Fi, and the unit then controls the driver 18 to vary the light output based on the communicated settings.

The overall system also comprises one or more data stores in the form of one or more databases 20, 22, 24. Database here can most generally refer to any size data structure from a small look-up table to a large database. In embodiments, these comprise an input-taste mapping database 20, a taste-light mapping database 22, and a taste compensation database 24. These will be discussed in more detail shortly, but briefly the input-taste mapping database 20 maps between the possible values that may be received via the input device(s) 6 and a corresponding change in taste in case of those input values arising, while the taste-light mapping database 22 maps between desired changes in taste and the corresponding light output effect required to achieve the respective change in taste. These may be used to create a third database 24, the taste compensation database 24 which maps directly between the input values and the lighting effects required to compensate or achieve a change in taste corresponding to the input.

The controller 4 is coupled to at least the taste compensation database 24, though in embodiments the controller 4 need not necessarily be coupled to access information directly from the input-taste mapping database 20 nor taste-light mapping database 22. In embodiments the controller 4 is located in the target environment 2 (along with the one or more lighting devices 8, 10 and one or more input devices 6) while the databases 20, 22, 24 are located remotely from the target environment 2, e.g. on a computer system of a system operator or designer. In embodiments the taste compensation database 24 is hosted on a server of the system operator or designer and the controller 4 is coupled to access the taste compensation database 24 via a network such as the internet. The input-taste mapping database 20 and taste-light mapping database 22 may be stored on the same server or another computer of a system designer or operator, and the taste compensation database 24 is created and potentially updated based on the input-taste mapping database 20 and taste-light mapping databases 22. The databases 20, 22, 24 are pre-configured but may also be updated at intervals or in an ongoing manner.

In alternative embodiments it is not excluded that the controller 4 has access to the input-taste mapping database 20 and taste-light mapping database 22 as well, e.g. via a network such as the internet; or that the controller 4 determines the mapping between input and light directly from the input-taste mapping database 20 and taste-light mapping database 22 without a third database 24 being created based on the two. In further embodiments, it is not excluded that one or more of the databases 20, 22, 24 are located locally at the controller 4 or in the target environment 2. However, it may be preferred to implement them remotely so that they can be updated centrally by the system operator, and also made available to multiple different instances of the controller 4 in multiple different environments 2. For example the taste compensation database 24 may be arranged to serve the lighting control in a number of different restaurants or homes.

As mentioned, varying the colour of ambient lighting has been shown to influence perceived taste in recent studies. Published research shows that certain taste attributes such as sweetness or fruitiness can be positively influenced by coloured ambient light—e.g. see references [1] and [2]. Different colour light can influence different perceived taste attributes, e.g. for wine it is estimated that red light can increase perceived sweetness levels by 60%. It is also established that the colour of the food or beverage, or its packaging, has an effect on taste—e.g. see references [3], [4] and [5]. Note that the changes in perceived taste due to ambient light are believed to be solely due to the ambient light, and independent of the effect of any colour change of the food or beverage which is being consumed.

Further, it is documented that the perceived taste of food or drink can vary according to a number of environmental properties. For example air pressure and humidity are both known to change perceived taste, and this has been validated through aerospace related use cases. Another example is the temperature of the environment. Other factors may also influence perceived taste. Accordingly the taste of food and drinks consumed in places where properties such as altitude, humidity and/or temperature vary from the norm, e.g. on board an aircraft, can vary significantly from those that a consumer may expect. For instance, the following references establish that higher taste and odour thresholds—with few exceptions—are generally observed at low pressures, as might be present on an aircraft flight.

[6] “Effect of Altitude on Taste Thresholds”, J A Maga and K Lorenz, 1972, Perceptual and Motor Skills, Volume 34, pp. 667-670 (http://www.amsciepub.com/doi/abs/10.2466/pms.1972.34.2.667)

[7] “Odor and Taste Perception at Normal and Low Atmospheric Pressure in a Simulated Aircraft Cabin”, Andrea Burdack-Freitag, Dino Bullinger, Florian Mayer and Klaus Breuer, Journal für Verbraucherschutz and Lebensmittelsicherheit, March 2011, Volume 6, Issue 1, pp. 95-109 (http://link.springer.com/article/10.1007/s00003-010-0630-y?LI=true)

This change in taste could be compensated for by the addition of extra quantities of certain ingredients such as salt, sugar and herbs; and a reduction in others, such as those which are sour. Consumers are however increasingly concerned about their weight and health, and today most larger food manufacturers are actively seeking ways of making their products more healthy whilst retaining the same “good” taste attributes—in many cases this means trying to reduce the sugar and salt content through finding healthier substitutes. Therefore adding more of these unhealthy ingredients in order to maintain the taste of food at changing altitudes or humidity levels goes against current health driven consumer attitudes, and may be a problem for food producers who want to produce good tasting food which is also healthy.

According to embodiments disclosed herein, the one or more input devices 6 comprise an atmospheric pressure sensor arranged to sense a change in pressure in the target environment 2, a humidity sensor arranged to sense a change in humidity in the target environment 2, a temperature sensor arranged to sense a change in temperature in the target environment 2, and/or another sensor arranged to sense some other measurable environment characteristic of the target environment 2. Based on these one or more inputs, the controller 4 varies the spectrum of illumination provided by a light source to compensate for any change in taste which may be experienced through the change in the characteristic which is being measured (or at least partially compensate). Parameters controlled by such a system may include colour and/or intensity of the ambient light and/or targeted light.

Thus the system tailors the spectrum of emitted light in order to influence taste in a target environment with known characteristics in response to one or more sensor inputs. For example, the altitude and humidity may be sensed in an aeroplane, and the spectrum of cabin or personal lights adjusted in dependence on the measured signal so as to compensate for the altitude and humidity induced taste changes. One benefit of this is that the same food taste can be maintained through changing the spectrum of illumination, reducing the need for additional sugar, etc. Another example may be applied to consumers who live at high altitudes, who may wish to achieve the same taste properties as others who live at lower altitudes. A further example may be applied to bathroom products such as toothpaste, where bathroom humidity may be used to control the light spectrum and therefore perceived taste of toothpaste or mouthwash, etc. In yet another example, the compensation may be applied in order to compensate for an effect of natural light on perceived taste—e.g. if the amount of daylight entering a room changes so as to affect a consumer's perceived taste, the amount of natural light entering or the artificial light in the room may be adjusted to compensate for this effect.

The desired light spectrum may be computed through two mappings which are established between (i) the desired taste attributes and the actual taste attributes in the context of the environment characteristics being measured, and (ii) a light spectrum required to generate a perceived change in taste for a given taste attribute and intensity.

In embodiments, the system may be implemented through the following steps.

(a) Characterise the desired taste compared to the actual taste of a product, under conditions of different input conditions that may be experienced in the target environment 2. This creates an “input to taste” mapping for each taste attribute (sweet, fruitiness etc.). For example, if a food loses 50% of its sweetness attribute at altitude X compared to ground level, then the desired taste at altitude X would include a taste whose sweetness is 50% greater than normal. An example output of this mapping could take the form example <taste attribute, compensation, altitude>, for example <sweetness, 50%, 30,000 ft>. These results may be stored in the input-taste mapping database 20.

(b) Establish the potential taste Influence of the lighting system. This means creating a “taste to light” mapping—i.e. specifying which potential colour (or spectrum distribution) can influence which taste attributes, and by how much. For example, the cited studies disclose that red ambient light may influence the perceived sweetness taste property by up to 60%. An example output of this mapping could be therefore be <red (intensity y), sweetness, 60%>. These results may be stored in the taste-light mapping database 22.

(c) Using the input-taste mapping database 20 and taste-light mapping database 22, compute which light settings are required to provide the user with the intended ‘neutral’ taste (i.e. no perceived taste changes) and store these in the taste compensation database 24.

(d) In operation of the lighting system in the target environment 2, measure one or more environmental characteristics (e.g. altitude, humidity etc.) which have been characterised in the databases, and uses the measurements of these characteristics to select an entry from the taste compensation database 24 so as to select a colour and intensity of light for a desired taste attribute compensation level.

An example use case is as follows. Making use of references such as [6] and [7], an airline specifies an altitude-to-taste mapping for a meal to be eaten at 30,000 ft which includes an indication that the sweetness attribute of the consumed meal is likely to reduce by 50% when at 30,000 ft. Further, making use of references such as [1] to [5], a lighting company has specified that an ambient light of luminosity 16 cd/m2 and with chromaticity values (CIE1931) of x=0.637 and y=0.322 can yield a perceived increase in sweetness of 60%. Assuming a linear relationship between luminosity and perceived sweetness threshold, it is determined that to provide the level of taste compensation desired by the airline, ambient lighting with a luminosity 13 cd/m2 and with chromaticity values (CIE1931) of x=0.637 and y=0.322 will be required. This is stored in the taste compensation database 24 and recalled and used to control the ambient lighting system when altitude sensors indicate the aircraft is flying at 30,000 ft and—optionally—when a meal is to be served.

According to other or further embodiments disclosed herein, properties of light may be used to control the taste of food in dependence on identifying a particular product being consumed in the target environment 2. Alternatively or additionally, properties of light may be used to tune the taste of food to the tastes of a particular user (e.g. consumer or chef) or group of users (e.g. tuned to the local tastes of consumers in a particular geographic region). Parameters controlled by such a system may again include colour and/or intensity of the ambient light and/or targeted light, both of which may be adapted to the product and/or personalized to the individual's or group's taste to light sensitivity.

It is known that perception of taste may be influenced by learned correlations, e.g. red food may be associated more with sweet than sour food. Using embodiments disclosed herein it is possible to enhance or amplify peoples' perception of a ‘learned’ taste, e.g. where it is desired to enhance the flavour of coloured products such as -fruit. Alternatively, it may be desired to shift the flavour away from the learned taste associated with the intrinsic colour of the product, e.g. to give people the impression that black coffee tastes sweeter by providing a red ambient. Or where these associations are not already present (e.g. for colourless products such as water), the technique may advantageously be used to create a taste for products for which people have no ‘learned’ taste.

In embodiments the one or more input devices 6 comprise means for identifying the product 14. This may comprise a detector for automatically detecting an identification of the product, e.g. from passive or active packaging of pre-packaged food so that data input or read from the packaging may be used by the system to control the ambient and/or task light setting(s). For instance the input device 6 may comprise a barcode reader for reading an identifier of the product 14 printed on its packaging in the form of a one or two dimensional barcode, or a sensor or image recognition algorithm for recognising the colour of the packaging or pattern or mark printed on the packaging. These are forms of passive packaging. As another example, the input device 6 may comprise an RF transducer for reading an RF tag associated with the product 14, e.g. again incorporated into its packaging. This is an example of active packaging. Products that could incorporate such active or passive packaging techniques include ready meals, coffee capsules, and many others. Alternatively or additionally the input device 6 may comprise a user interface by which a user can manually enter an identifier of the product 14.

In such embodiments, instead of (or as well as) mapping between environmental factors and taste, the input-taste mapping database 20 comprises a mapping between an identifier for one or more products 14 and a taste objective for each respective product. Accordingly, the taste compensation database 24 comprises a mapping between product identifier and light output required to create the corresponding influence on taste specified for the respective product. The controller 4 is arranged to access the taste compensation database 24 and control the light output in the target environment 2 accordingly. For example, the database 24 may be implemented as a public database from which users can access the relevant lighting effects for the products they are consuming, e.g. accessing the database over network such as the internet.

As an example use case, a user buys a low-salt sweet & sour chicken ready meal made by a certain manufacturer. The user scans the barcode using the camera on his or her smart phone, which thereby detects an identifier of the ready meal. Alternatively the user could enter an identification manually via the touchscreen of the phone, or an RF ID tag could be read by an internal or external transducer. An application running on the smart phone implements at least some of the functionality of the controller 4, and the camera or touchscreen provides the input device 6. The application uses the determined identifier of the ready meal to query a remote server hosting the taste compensation database 24. The taste compensation database 24 comprises an indication of certain light settings designed to compensate for the low salt content of the ready meal by increasing the user's perceived sensation of saltiness, and this information is returned to the application on the smart phone. The application then communicates these settings to the driver 18 of the lighting system via a suitable interface such as a wireless interface, e.g. Bluetooth or Wi-Fi (and via any part of the controller 4 implemented in a separate unit installed in the environment 2 if required), in order to tune the colour and potentially other properties of the light and thereby enhance the flavours which are important to the meal, including compensating for the low salt content.

In another use case a user buys coffee capsules for use in a coffee machine, where a barcode or RF ID tag is included on each capsule. The coffee machine is networked to the lighting control system, and also to a remote server. When the capsule is inserted into the machine, the coffee type is communicated to the server which returns the lighting attributes most suited to the users taste objective or profile for the specific capsule.

In further alternative or additional embodiments, the one or more input devices 6 comprise means for identifying a user. This may comprise a detector for automatically detecting an identification of the user, e.g. a facial recognition algorithm, or an RF transducer for reading an RF card or other tag of the user. Alternatively the means may comprise a user interface by which a user can manually enter an identification.

In these embodiments, the input-taste mapping database 20 comprises a mapping between an identifier for one or more users and a taste objective for each respective user. Accordingly, the taste compensation database 24 comprises a mapping between user identifier and light output required to create the corresponding influence on taste for the respective user. The controller 4 is arranged to access the taste compensation database 24 and control the light output in the target environment 2 accordingly.

The user being identified could be the consumer of the product in question, e.g. a customer in a restaurant or a consumer of a product bought from a shop. In this case the light effect may be provided to compensate for the particular sensitivity of that user to one or more taste attributes. For example if a user has a weaker sense of sweetness, a corresponding light effect may be provided to compensate for this by increasing perceived sense of sweetness. Instead of compensating for a weaker sense, the light effect may also be used to emphasize personal preferences in tastes. For example, the same product could be made to taste more sweet for a particular person and less sweet for another person by applying different light conditions. Alternatively the user being identified could be a provider of the product in question, e.g. a chef. In this case the light effect may correspond to a taste effect desired to be created by the chef.

Again the controller 4 may be implemented at least partially in an application running on a smart phone or other user terminal, and the camera or user interface of the phone or terminal may provide the input device 6. The taste compensation database 24 may be implemented in a central database storing information for multiple users, or may be implemented locally on the phone or terminal. For example in one embodiment, the user may maintain the input-taste mapping database 20 and taste compensation database 24 locally at his or her own user terminal, while the taste-light mapping database 22 may be hosted centrally and accessible by other users.

In an example use case, a user buys a certain brand of toothpaste and scans the barcode with a smart phone. Profile information suggests this user likes sweet foods. The bathroom lighting system characteristics are then controlled such that the properties of the light support a sweeter taste for the toothpaste.

In further alternative or additional embodiments, the one or more input devices 6 comprise means for identifying a location of the target environment 2. This may comprise a geographic location technology such as GPS for automatically detecting location, or a user interface allowing a user to manually indicate the location. Again the controller 4 may be implemented partially in an application running on a smart phone or other user terminal, and the location technology or user interface of the phone or terminal may provide the input device 6.

In such embodiments, the input-taste mapping database 20 comprises a mapping between one or more locations and a taste objective for each respective location. Accordingly, the taste compensation database 24 comprises a mapping between location identifier and light output required to create the corresponding influence on taste specified for the respective location. The controller 4 is arranged to access the taste compensation database 24 and control the light output in the target environment 2 accordingly. The databases 20, 22 and/or 24 may be accessed from a public or central source, or may be maintained locally at the controller 4 in the target environment 2 (e.g. on a user terminal).

This variant may be used to provide region specific taste compensation through light. Differences in regional trends or tendencies sometimes exist in users' perceived taste, and also differences in the influence the intensity of light has on the taste threshold for various taste attributes (sweet, bitter, etc.). Therefore in embodiments, the system may be configured by determining attributes of a product to be consumed, determining region specific taste preferences of users in the relevant region, and determining a corresponding light setting to achieve the specific taste preference (colour and/or intensity). The controller 4 then uses the databases to calculate regional taste compensation settings, and apply these to the lighting control system.

In further alternative or additional embodiments, the one or more input devices 6 comprise a user interface allowing a user to explicitly specify a desired taste objective. For example the user may explicitly enter an input to the effect “increase sweetness” or “increase sweetness by 50%”, or similarly for other taste attributes such as saltiness, sourness and/or bitterness.

In this case, the controller 4 may directly use the taste-light mapping database 22 to look up the light settings for creating the taste objective specified by the user, e.g. the increase in red light that will create a certain increase in sweetness. In such embodiments, the other databases 20, 24 are not necessarily required, unless the system is also configured to control taste based on other inputs such as environmental factors as well. The taste-light mapping database 22 may be implemented at a public or central source so as to be accessible by multiple different users in different environments. Again at least part of the controller 4 may be implemented in a user terminal such as a smart phone, with the user interface of the phone or other terminal providing the input device 6. The user terminal looks up the required light settings in the taste-light mapping database 22, and then communicates the settings to the driver 18 via a suitable interface such as a wireless interface, e.g. Bluetooth or Wi-Fi (and any part of the controller implemented in a separate unit installed in the environment 2).

According to various embodiments discussed above, there is thus provided a system which uses the properties of ambient light and/or targeted light to control the taste of food.

In embodiments, different combinations of inputs may be used. For example, the system may be configured to adapt based on environmental factors such as altitude or humidity, and/or based on the identity of the product 14 being consumed, while the behaviour of the system may be also personalized to the individual's taste to light sensitivity.

In embodiments, the light may be controlled from a personal device (e.g. smartphone) providing information on the identity of the user, location of the user in the space and the user's taste preferences. Further, these personal preferences may be used to add onto a preferred setting preset by, for example, the restaurant's chef and associated with the dish which is served, as a kind of personal fine tuning or modulation of taste.

One advantage that may be achieved in embodiments is to personalise the taste of food without the need for adding salt and pepper to the food. Another possible advantage is for restaurants to apply an ambient light setting which improves peoples' experience in tasting and appreciating food. A further advantage may occur when the invention is incorporated with a system to determine the altitude of a user, and through a suitable model, to provide ambient light settings which go some way to compensate for the change in taste which occurs at increasingly altitudes. Further advantages may be achieved by compensating for changes in other environmental factors such as air pressure generally, temperature or natural light.

With regard to the databases 20, 22, 24, these may be preconfigured at a design stage, for example based on research such as presented in references [1] to [7]. The association between taste and light, and between input values and taste (e.g. between altitude, humidity or geographical region and taste), may be determined based on surveys of suitably large samples of test subjects. For example the effect on taste may be quantified in terms of the subjects' reported rating or scoring of their perception of one or more taste attributes; or based on a perceptual metric such as the “just noticeable difference” (JND) or perception threshold, i.e. the smallest amount by which a property (e.g. light, altitude, humidity etc.) needs to be varied in order for the subject to report a perceived difference in the taste attribute. Suitable ways of measuring perception are disclosed in the cited references. The taste attributes in question may be expressed in terms of one or more of: sweetness, saltiness, bitterness and/or sourness, or others such as perceived “fruitiness”, or combinations of these. Also, note that while the associations have been described above in terms of a mapping in a database, it is not excluded that they could be modelled as an analytical relationship (i.e. a formula or function).

In embodiments the controller 4 may be pre-configured with the results of the taste-compensation database 24. Alternatively, the controller 4 may be configured to access the taste compensation database 24 from a central or public data source such as a server of the system designer or operator. An advantage of a central or public database is that the designer or operator can update the database over time as new data becomes available, and the controller 4 will then continue to operate based on the updated data. I.e. the database can continue to be updated after installation and deployment of the controller 4 and/or lighting system. Similar comments may be made in embodiments where the controller directly accesses the light-taste mapping database 22 (e.g. to service a user's explicit request for a change in taste attribute), and/or directly accesses the input-taste database 20 (e.g. if the controller 4 computes the input to light output association itself on a “case-by-case” basis or “on the fly” based on the input-taste database 20 and light-taste mapping database 22 instead of relying on the results being pre-combined into a third database 24).

In one particularly advantageous embodiment, the system is configured to allow crowdsourcing of new data. Whilst some information for the input-taste mapping database 20 and taste-light mapping database 22 can be pre-configured based on existing research, it may be desirable to augment the existing data. Accordingly, embodiments of the present disclosure provide a participatory sensing system using crowdsourcing techniques, whereby consumers can provide feedback on their perception of the effect colour has on taste, and/or the effect of other factors such as altitude and humidity. These can be aggregated together over time, and combined with sensor data (for example altitude, humidity) to automatically generate and/or update the input-taste mapping data and/or the taste-light mapping data.

For example, when a user consumes a product 14 in certain light conditions or under differing light conditions, the user may report on his or her perception of taste along with an indication of the relevant light settings from the controller 4. The controller 4 may be at least partially implemented by an application on a mobile terminal or other user terminal, or the controller 4 could report the settings to the application. The user may then choose to submit a perception report to a server of the designer or operator via his application and a network such as the internet. The application may automatically include the light settings in the report. Other factors such as an identification of the product 14 may also be included in the report, either manually or by an automatic detection (e.g. using a barcode or RF tag). As reports from many users are aggregated centrally by the system operator over time, these can be used to update the taste-light database 22 and taste compensation database 24, and potentially continue to update them as further reports come in.

Alternatively or additionally, when a user consumes a product under influence of one or more environmental factors such as altitude or humidity or under differing values of such factors, he or she may use the application to submit a report of perceived taste. The application may automatically detect the sensor reading or receive the sensor reading from a separate control unit, and automatically include this information in the report to the operator or designer. As reports from many users are aggregated centrally by the system operator over time, these can be used to update the input-taste database 20 and taste compensation database 24, and potentially continue to update them as further reports come in.

In further embodiments, the system exploits a difference between the influence of ambient lighting on taste and the influence of actual colour on taste.

As already stated, the effect of ambient light on taste has been found by researchers to be independent of the effect the colour of a food or beverage has on taste—see references [1] and [2]. It is true that in many cases the perceived colour of food or beverages may be influenced by the characteristics of the ambient light. For example a white or transparent product 14 will itself appear redder in presence of red ambient light. As can be seen from references [3]-[5], the colour of a food can significantly influence its taste too. Accordingly the taste of foods or beverages which have a white or transparent colour may be influenced by the colour of ambient light, which in turn influences the colour of the food or beverage. While there can be some divergence with colours such as blue, in general there is a correlation between the effects of ambient light and the effects of actual food or beverage colour (for example, red generally results in a perceived sweeter taste). Nonetheless, the treatment of perceived taste changes through ambient light colour and perceived taste changes through actual food colour do offer two independent ways of varying perceived taste.

Hence in embodiments, the lighting system comprises a primary lighting system comprising one or more primary lighting devices 8, and a secondary lighting system comprising one or more secondary lighting devices 10.

The primary lighting 8 provides the general ambient lighting, e.g. the one or more primary lighting devices 8 may comprise one or more wall washers or uplighters. The controller 4 may be configured to control the light output by the primary lighting 8 in order to control the colour and/or intensity of the ambient light. The secondary lighting 10 provides light output arranged to influence the actual food/beverage colour of the product being consumed, e.g. by a spotlight or other task light targeted on a location from which the product 14 is to be consumed (e.g. a spot on the table 12). The controller 4 may be configured to control the light output by the secondary lighting 10 in order to control the colour and/or intensity of the light reflected from the product, and therefore the appearance of the product 14. In an application, the primary lighting 8 could thus be used to influence the perceived taste through ambient lighting and the secondary lighting 10 could be used to influence the perceived taste through coloured target lighting on the product being consumed. In this case the primary lighting 8 and secondary lighting 10 are operated towards the same taste attribute. However, the primary, ambient lighting 8 could also be used to influence perceived taste while the secondary, target lighting 10, independently from the primary, ambient lighting 8 could be used to improve appetite or attractiveness of the product being consumed.

The controller 4 may be configured to control one or both of the primary, ambient lighting 8 or the secondary, target lighting 10 in order to influence the taste based on the relevant input, e.g. to compensate for the effect of altitude or humidity. An example of this could be influencing the taste of a toothpaste according to bathroom humidity by varying the colour of the ambient light. In embodiments, the controller 4 is configured to control the primary, ambient lighting 8 independently of the secondary, target lighting 10, or vice versa, or to control both independently of one another. That is, the controller 4 can vary the light output of the primary, ambient lighting 8 without necessarily having to vary the light output of the secondary, target lighting 10, and/or vice versa; and where it does vary both, the change in the light output of one is not necessarily linked to or dependent on the light output of the other. The behaviour of the different types of lighting 8, 10 required to achieve the desired effect may for example be determined using crowdsourced inputs to gather further information, as discussed above, and/or a combination of the results from existing research such as references [1]-[7].

For instance, if red light is being used to sweeten a cup of coffee, this could be performed using a red light targeted at the cup of coffee in a way that, say, the colour of the wider table or the walls is not unduly reddened also (which a user may not prefer for other reasons). On the other hand it may be that in circumstances, the desired influence is most effectively achieved using ambient light, and is preferred for that reason. In embodiments, a combination of the two types of lighting could also be used to balance different considerations such as attractiveness of a product and taste of a product.

In embodiments, ambient light may have a dual functionality, i.e. creating an atmosphere on the one hand and influencing peoples' taste on the other hand. In one embodiment, the ambient lighting system 10 (e.g. a wall washer) may create a proper dining atmosphere and the task lighting system 10 (e.g. a spot light) may create a taste influencing task light onto the dish or other food or drink. Or vice versa, the task lighting 10 may be used to create an aesthetic effect on the dish while the ambient light is controlled to influence the perception of taste. In further embodiments, the effect of a taste influencing task light may be enhanced by providing a contrasting or complementary ambient light condition such that the task light is more pronounced, or vice versa a task light 10 may be used to contrast or complement the effect of taste influencing ambient lighting.

It will be appreciated that the embodiments above have been described only by way of example. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A system for influencing a being's perception of taste of a product during consumption, the system comprising:
one or more input devices arranged to receive an input, the input corresponding to a desired change in taste of the product during consumption;
one or more lighting devices arranged to provide a light output in a target environment; and
a controller configured to control the light output, in dependence on said input, to influence the being's perception of taste of the product during consumption in said target environment to achieve said desired change in taste,
wherein said input further comprises an identification of a user, a location and/or product and wherein the controller is configured to control the light output further based on the identification.

2. The system of claim 1, wherein the one or more input devices comprise one or more sensors arranged to take a measurement of one or more environmental variables from the target environment, the one or more environmental variables causing a perceived change in the being's taste; and
the controller is configured to control the light output, in dependence on said measurement, to produce an influence on the being's perceived taste at least partially compensating for said perceived change.

3. The system of claim 2, wherein the one or more environmental variables comprise one or more of: an altitude of the target environment, an air pressure in the target environment, a humidity in the target environment, a temperature in the target environment, and/or natural light in the target environment.

4. (canceled)

5. The system of claim 1, wherein the input device comprises a user interface arranged to receive a user preference explicitly specifying a change in taste; and
the controller is configured, in dependence on the user preference, to control the light output to influence the being's perceived taste at least partially toward the specified change.

6. The system of claim 1, wherein the lighting devices comprise an ambient lighting device arranged to provide an ambient light output, and a target lighting device arranged to provide a light output targeted on a location from which the product is to be consumed; and
the controller is configured to produce said influence on the being's perceived taste by controlling the light output of at least one of the ambient lighting device or the target lighting device.

7. The system of claim 6, wherein the controller is configured to produce said influence on the being's perceived taste by controlling the light output of at least one of the ambient lighting device or the target lighting device independently of the other.

8. The system of claim 6, wherein the controller is configured to produce said influence on the being's perceived taste by controlling the light output of both the ambient lighting device and the target lighting device.

9. The system of claim 1, wherein the controller is configured to determine the light output based on an association between a desired change in taste of a product and a light output, provided by a remote data store, by accessing the data store over a network.

10. The system of claim 1, comprising:
a first data store providing an association between a change in taste of a product and an input received from an input device;
a second data store providing an association between a light output and a change in taste of a product; and
a third data store providing an association between a light output and an input received from an input device, based on the associations provided by the first and second data stores;
wherein the controller is configured to determine the light output corresponding to said change in taste of said product based on the association provided by the third data store.

11. The system of claim 10, wherein the first, second and/or third data stores are implemented remotely from the target environment and controller, and the controller is configured to access the association provided by at least one of the data stores via a network.

112. The system of claim 9, wherein the association provided by at least one of said data stores is kept updated over time, and made available to be accessed by said controller and other instances of the controller in other target environments.

13. The system of claim 9, wherein the association provided by at least one of the data stores is updated based on user feedback of perceived taste, the feedback being received over a network.

14. A method of influencing a being's perceived taste of a product during consumption, the method comprising:
receiving an input from one or more input devices, the input corresponding to a desires change in taste of the product during consumption; and
in dependence on the input, controlling a light output of one or more lighting devices in a target environment to influence the being's perceived taste of the product during consumption,
wherein the method further comprises receiving an identification of a user, a location and/or a product from the one or more input device and controlling the light output further based on the received identification.

15. A computer program product for influencing a being's perceived taste of a product during consumption, the computer program product comprising code embodied on a computer readable storage medium and configured so as when executed on processing apparatus to perform operations of:
receiving an input from one or more input devices, the input corresponding to a desired change in taste of the product during consumption;
receiving an identification of a user, a location and/or a product from the one or more input device; and
in dependence on the desired change in taste of the product during consumption and the identification, controlling a light output of one or more lighting devices in a target environment to influence the being's perceived taste of the product during consumption.
